(19) United States
(12) Patent Application Publication
(10) Pub. No.: US 2005/0182430 A1
(43) Pub. Date: Aug. 18, 2005

(54) Anastomosis device and method

(76) Inventor: Robert R. Schenck, Chicago, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/056,650

(22) Filed: Feb. 11, 2005

Related U.S. Application Data
(60) Provisional application No. 60/543,890, filed on Feb. 12, 2004.

(51) Int. Cl. A61B 17/08

(52) U.S. Cl. 606/153

(57) ABSTRACT

An anastomosis ring for interconnecting ends of living vessels in apposition of the ring having a tubular body with an annular wall surrounding a hollow bore. A plurality of impaling projections are provided at one end of the tubular member and a plurality of impaling projections are provided at the other end of the tubular member to impale a vessel end. A coupler device with a detachable connection holds the anastomosis ring adjacent the severed vessel ends. A first vessel end is inserted through a hollow bore of the tubular member and everted and impaled on the impaling projections. The second vessel end is drawn over the everted first vessel end and the intimas of the respective vessel ends are being brought into apposition and the second vessel and is impaled on the impaling projections. The anastomosis ring is then disconnected from the coupler device.

### CROSS-REFERENCE TO RELATED APPLICATION

This application claims the benefit of U.S. Provisional application Ser. No. 60/543,890, filed Feb. 12, 2004, which is incorporated by reference in its entirety herein.

### FIELD OF THE INVENTION

The present invention is directed to an anastomosis device and methods for anastomosing ends of living vessels and, more particularly, to sutureless anastomosing in which an anastomosis tubular member is employed for anastomosing living vessel ends.

### BACKGROUND OF THE INVENTION

A wide variety of anastomosis devices and methods have been developed for anastomosing ends of living vessels. One known procedure for anastomosing blood vessels includes the use of sutures, i.e., stitches. One known shortcoming with conventional suturing techniques is their tendency to be relatively tedious, particularly with blood vessels of small diameters, such as vessels being one millimeter or less in diameter. Experience has revealed that this is especially the case when anastomosing children's vessels because they are even smaller and prone to spasm. In addition, successful anastomosing of blood vessels is highly dependent on the proper placement of the sutures by the surgeon. Thus, in certain instances, such known conventional suturing anastomosing techniques are time-consuming to a point that undesirably extends the duration of a surgical procedure.

With the exception of the aorta and vena cava, human blood vessels have a naturally occurring contractility known as circumferential compressive stress which resists dilation. As the vessel diameter decreases and the relative wall thickness increases, these compressive forces become proportionately larger. Radial tethering forces of tissues do exist around the vessel, but these are of lesser significance than longitudinal vessel motion tethering. It is therefore important to consider these forces to assure patency, i.e., the vessels ability to conduct blood flow, at the anastomosis site.

Even successful suturing of blood vessels does not assure continued patency at the sutured site. It is known that clotting of blood, known as “thrombosis,” may act to block blood flow through an anastomosed vessel. In addition to the already mentioned forces, thrombosis after microvascular repair may be caused by a number of other factors which include inaccurate placement of sutures, vessel spasms, stenosis and microclamp damage. Furthermore, it has been found that continuity of flow during the first twenty minutes after anastomosis is critical in preventing thrombus formation and that platelet aggregation, and later resolution, occurs in the first several hours after a microvascular anastomosis. Hence, it is critical that anastomosing procedures employ effective devices and be performed relatively quickly to relieve the procedure of the above-mentioned shortcomings. For example, it takes considerable time using multiple sutures, typically 6 to 10 sutures, when doing a replantation of each of multiple vessels for multiple digits. Thus, there is a need for a quicker anastomosis procedure which will shorten the operating time.

To aid in anastomosing blood vessels, a number of implantable devices have been employed at the anastomosis site for assisting to interconnect severed ends of blood vessels. Such devices, and methods for employing the same are disclosed in Schenck, U.S. Pat. Nos. 4,693,249 and 5,486,189. A number of other devices have been proposed, in various patents and articles, which include an external ring to which a pair of vessels having prepared openings may be tethered with a ring maintaining the lumen of the vessels in expanded condition at the anastomosis site which tends to provide assurance of patency. Some of these proposals in the prior art use anastomosis rings with radially outwardly extending protrusions for impaling the vessel ends and thereby securing them in apposition at the ring. In these situations one end of the first vessel is initially inserted through the hollow opening of the ring and then everted around and over the outside of the ring and impaled on the radial protrusions, and then, the end of the second vessel is drawn over the inverted first vessel end and also impaled on the radial protrusions with the intima of the vessel ends being engaged.

The present invention will be he reinafter described in connection with the preferred embodiments which are very small anastomosis rings suitable for very small vessels although the vessels could be and rings could be larger in size. It is the very small rings and very small vessels that are the most difficult and time consuming to provide the patency at the anastomosis site to prevent thrombosis at the site. Particular problem has been in the manufacturing or designing and providing a suitable anastomosis ring of this very small size with the outwardly extending protrusions thereon. By way of an example given and described in the illustrated embodiment herein, the tubular devices are only about 1.25 mm in inner diameter and 1.375 mm in outer diameter having about 0.125 mm wall thickness. The longitudinal extent of the ring or the width of the ring is only 0.5 to 1.0 mm in length. Manifestly, the rings may come in various seizes for different sizes of vessels being anastomosed together.

In addition to the difficulty in manufacturing, it is important to provide a quick and easy method of use for these very small vessels. There is a need to be able to hold the ring in position and to clamp or otherwise hold the first and second vessel ends so that the surgeon is able to use both hands to pass the first vessel through the opening and to evert it, to impale it and to be able to pull the second vessel end over the outside of the ring and impale it on the protrusions with the intima in engagement with each other.

Thus, there is a need to provide a new and improved anastomosis device and a method of manufacture thereof as well as a manner of using the anastomosis device by a surgeon anastomosing vessel ends.

### SUMMARY OF THE INVENTION

In accordance with the illustrated embodiment, there is a new and improved anastomosis device having a tubular body with impaling protrusions formed thereon for interconnecting the ends of living vessels and to hold the same to assure patency of blood flow, for example, through the lumens of the vessel. This is achieved by an anastomosis ring which is formed with a tubular member having a plurality of impaling projections at one end of the tubular body and a plurality of impaling projections at the other end of the tubular body. A curved wire is secured to an end wall of the tubular member and has at least one pointed impaling projection on the wire extending outwardly for impaling the inverted vessel and impaling the second end of the second vessel which is having its intima in engagement with the first impaled end. In the illustrated embodiment, a:pair of curved wires with pointed impaling projections are secured to each of the opposite end walls of the tubular member. The impaling projections are preferably spaced at 90° from each other and all face forwardly in one direction.

In accordance with this embodiment, each curved wire is secured, such as by welding, to an end wall of the very small tubular member. For example, the tubular member may be a cut piece of a cylindrical metal tubing or cylindrical plastic tubing having the very small outer dimension of about 1.25 mm and an overall length of, for example, 0.5 to 1.0 mm in longitudinal extent. Preferably the wires are curved and the ends thereof are bent outwardly to form the impaling ends or spokes which inscribe a circle larger in diameter than that of the ring diameter. By way of example only, the illustrated impaling protrusions have a length of about 0.4 mm to 1.0 mm and extend outwardly at an angle of about 45° to the axis of the ring. The ends of the impaling protrusions, in the illustrated embodiment, inscribe a circle of about 2 mm in diameter.

In accordance with the illustrated embodiment, the cut tubular members are formed with slots at one end wall to receive the corner of the curved wires where the wire, impaling protrusions or spikes are bent outwardly from the end of the tubular member.

The manufacturing process of these particular rings involves the cutting of the tubing for the ring into segments, slotting the tubing segments at an end, cutting the wire to length and spinning the cut wire segments to have a curve with a diameter to match that of the tubular segments and to grind the impaling end of the wire into a point. The curved wire is welded to the end wall of the tube segment, such as by a laser spot weld and then the protrusions or spikes are bent or otherwise positioned at the correct angle. If there is any excessive wire, the excess is cut from the curved wire.

In accordance with a further aspect of the embodiment herein, the method of anastomosis first and second vessel ends comprises providing the anastomosis ring with its tubular body and attached wires providing impaling projections at ends of the ring, detachably holding the anastomosis ring with a coupler device so that it is positioned adjacent the first and second vessel ends and is held in position allowing the surgeon to have both hands free, and extending the first vessel end through the hollow bore of the tubular body and everting the first vessel end over the exterior surface of the tubular body, impaling the first vessel end on the impaling projections of the anastomosis ring, drawing the second vessel end over the inverted first vessel end and bringing the intimas into apposition, and separating the anastomosis ring from the anastomosis coupler thereby allowing the anastomosis ring to remain with the living vessels and without being attached to the coupler device.

In the preferred and illustrated embodiment, the first and second vessel ends are held by first and second clamps on the coupler device in position adjacent the anastomosis ring to assist in allowing the surgeon to quickly grasp each of the vessel ends and move them and to impale them on the anastomosis ring.

In the preferred embodiment, the anastomosis ring attachment to the anastomosis coupling is by a breakable tab or a suitable, releasable adhesion.

### BRIEF DESCRIPTION OF THE DRAWINGS

The present invention will be described hereinafter in connection with the illustrated embodiment wherein:

FIG. 1 is a perspective view of an anastomosis ring illustrating the tubular body with a curved ring attached thereto having protrusions projecting outwardly to impale vessel ends;

FIG. 2 is a perspective view of two bent wire half-rings which will be secured,.such as by laser welding, to an end wall to form the anastomosis ring of FIG. 1;

FIG. 3 is an enlarged perspective view of a small, tubular body prior to having the curved wires of FIG. 2 attached thereto;

FIG. 4 illustrates an embodiment of the invention having a pair of curved rings each secured to one of the opposite end walls of the anastomosis ring to provide protrusions at each of the opposite ends of the ring for impaling the vessel ends;

FIG. 5 is a perspective diagrammatic view of a coupler device used for holding an anastomosis ring and for gripping the first and second vessel ends;

FIG. 6 is a cross-sectional view taken along the line 6-6 of FIG. 5 showing the ring holding portion of the coupler device of FIG. 5;

FIG. 7 is an enlarged diagrammatic view of the vessel ends being impaled on the spiked protrusions and held with their intima in engagement using the preferred anastomosis ring;

FIG. 8 is a perspective view of an alternative anastomosis ring illustrating the tubular body attached to an exemplary ring holding device;

FIG. 9 is a perspective view of the anastomosis ring of FIG. 8 shown connected to a coupler device and having the vessel ends in partial cross section and impaled on the spiked protrusions;

FIG. 10 is an enlarged side elevational view of the anastomosis ring and coupler device of FIG. 9 shown with the vessel ends in partial cross section and impaled on the spiked protrusions; and

FIG. 11 is a perspective view of the anastomosis ring of FIG. 8 connected to a coupler device, the anastomosis ring is shown prior to being detachably separated from the exemplary ring holding device.

### DETAILED DESCRIPTION OF THE PREFERRED EMBODIMENT

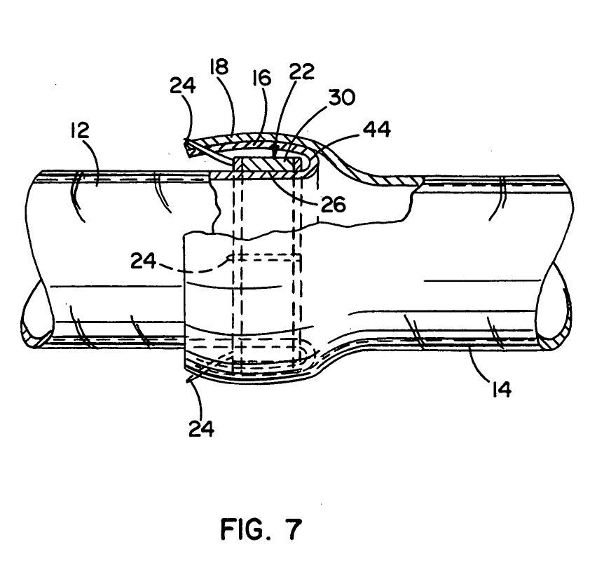

In the illustrated embodiment shown herein, there is provided an anastomosis device 10 for interconnecting a first living vessel, such as a blood vessel 12 to a second living vessel 14 which is often a severed portion of the same vessel as the first vessel 12. The anastomosis device is generally ring-shaped and includes a tubular body 22. Herein, the tubular body is cylindrical with a central bore or opening 28 through which is passed a first proximal vessel end 16 as shown in FIG. 7 which is then everted back over the top of the ring tubular body 22 and then the distal end 18 of the second vessel 14 is drawn over the inverted proximal end 16. The anastomosis ring includes pointed, impaling protrusions or impaling projections which are designed to impale the vessel ends 16 and 18 in position about the ring such that the patency of the vessel is maintained for blood flow through the bore 28 of the ring in this example.

The particular tubular body 22 is made from a biological, compatible material such as a metal stainless steel titanium or the like or may be formed of a suitable organic composition such as polyglycloic/polyactic. The preferred rings are usually obtained as tubes or tubing and are cut into segments and have an exterior surface 30 and an interior surface 26 which define an annular wall about the central opening 28. On the other hand, the ring bodies could be laser cut to form very small tubular members from a sheet of material rather than having been segmented from a preformed tube. Usually the exterior diameter of the ring at the exterior surface 30 is slightly larger than the relaxed exterior diameter of the vessel ends being joined. This results in the anastomosis vessel ends being radially stressed outward along the exterior surface of the anastomosis ring. Preferably the interior diameter of the ring is substantially equal to the natural, relaxed exterior, diameter of the vessel ends, if they were filled with blood. Thus, the diameter of the vessel passageway is maintained between the inverted ends secured to the ring-like body to insure the patency of the anastomosis site.

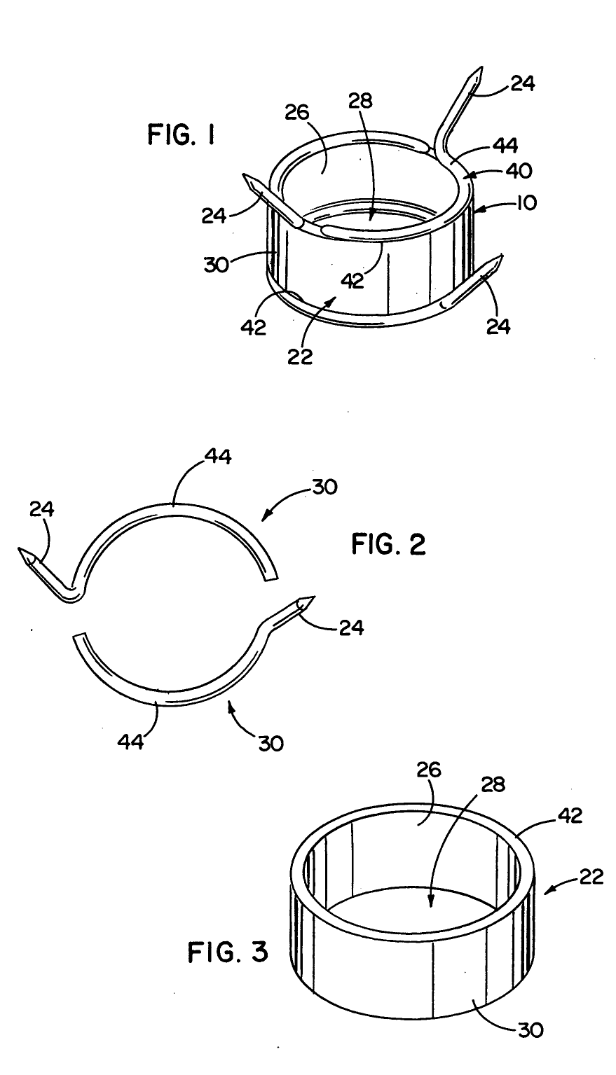

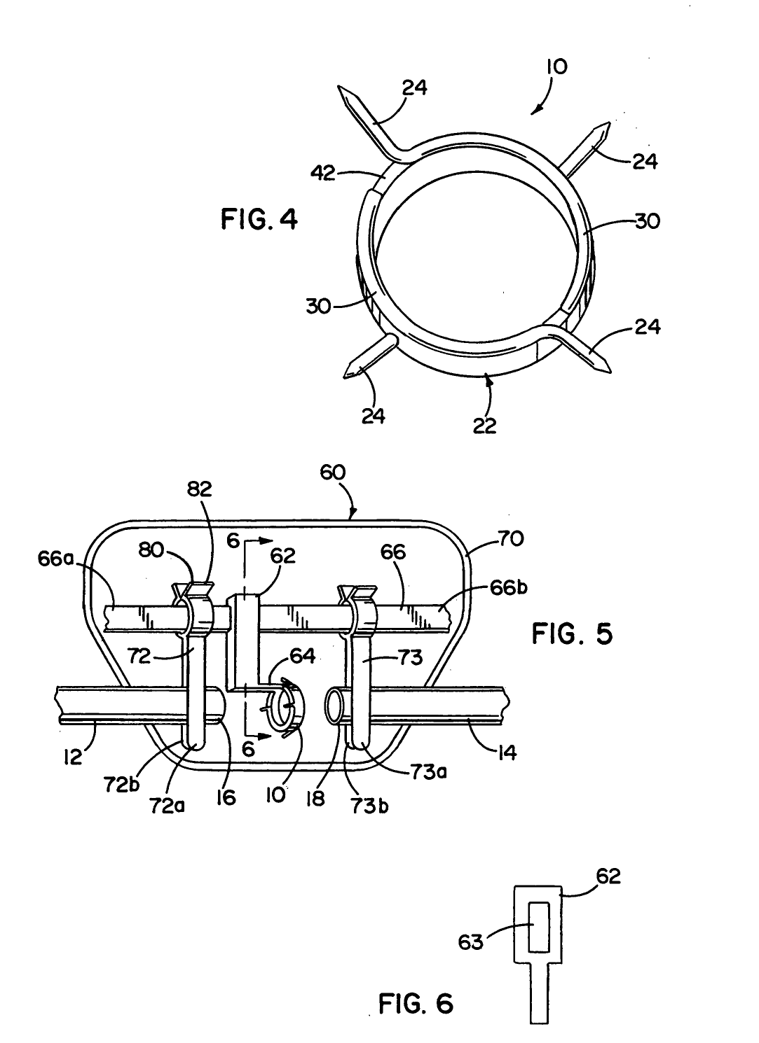

The anastomosis ring 10 is made with a multiple piece construction and comprises the tubular body 22 as well as a anastomosis curved wires 40 secured, as by laser welding, to an end wall 42 of the tubular body. The curved wire is preferably shaped and formed so as to be an extension of the tubular wall of the tubular member. That is, it is preferred that the radius of the curved portion 44 of the curved wire 40 have the same radius as that of the tubular body or member 22 and that the thickness of the wire approximate the thickness of the annular end wall of the tubular member 22. In the illustrated embodiment, the tubular member 22 was formed by cutting a metal tubing that was 1.25 mm in diameter with a 0.125 inch wall thickness. Herein, the curved wire 40 had a 0.125 mm diameter substantially matching the thickness of the annular ring wall. The wire was cut to length and then the wire segment was formed into the curve to have the same radius of curvature as that of the annular end wall. At least one end of the curved wire has a protrusion 24 integrally bent to extend radially upwardly and outwardly from the curved portion 44 to which it is integrally attached. Preferably when the wire segment is curved, the point is also bent outwardly and is ground into a point. After positioning the curved wire with a point 24 thereon at the end wall of the tubular member, the curved wire is then spot welded, as by a laser spot welding, to the end wall 42 of the tubular body 22 and the protrusion points are bent to the correct angle which is in this instance about 45°, although this angle may be varied. That is the angle of the illustrated impaling protrusions 24 is about 45° to the axis of the tubular body 22. These very small protrusions 24 are preferably ground to sharp pointed ends which are similar to a surgical needle in this illustrated embodiment of the invention. Thus, they easily impale the vessel ends. The protrusions project at the 45° angles to hold the impaled ends against being pulled apart. This 45° angle may be varied. In the embodiment of the invention illustrated in FIG. 2, there are shown two ring-like members having curved portions 44 and one protrusion 24 at one end thereof. Each of the curved bodies extends substantially less than about 180° so that they may be each positioned on the end of the wall end and laser welded thereto as best seen in FIG. 4.

In the illustrated embodiment, the anastomosis ring 10 has at each end, two projections 24 which are evenly spaced at about 180° apart. Manifestly, the projections 24 may be positioned at other spacings than 180° and there may be more than two, for example, three or four impaling projections 24 at each end of the device particularly for larger vessel sizes than that described for the illustrated embodiment of the invention. Usually it is preferred to space the projections 24 evenly in a circumferential direction; hence where there are two projections they will be spaced at about 180°. If there are three projections, they will be spaced at 120° apart and for four projections they will be spaced approximately 90° apart. Herein, the curved wires at the end wall of the ring are spaced 180° apart and the impaling projections on one are shifted 90° relative to the impaling projection at the other end wall. This results in a 90° circumferential spacing between the protrusions 24 on one end and the protrusions 24a on the other end of the tubular body for the embodiment illustrated in FIG. 4.

In accordance with the preferred method of holding the anastomosis ring 10 in position while the vessel ends 16 and 18 are being everted and impaled on the projections 24, it is preferred to provide a device such as a coupler device 60 (FIG. 5), which comprises a ring-holding device 62 to which is detachably secured the anastomosis ring device 10 with a breakaway connection such as tab 64 or a glued connection between the tab 64 and the ring. The ring-holding device is, as best seen in FIGS. 5 and 6, has a central rectangular slot 63 to mount it on a similarly shaped and sized, longitudinally extending bar 66 of the device . . . The ring holding device may be either stationery or it may slide on the bar. The bar may be fastened in a suitable frame 70, as best seen in FIG. 5 at its opposite ends in 66a and 66b. Thus, the anastomosis ring 10 is stabilized and held independently of the surgeon's hands so that the vessel 12 can be gripped at its end 16, as with a jeweler's forceps and advanced through the ring bore.

In the illustrated and preferred coupling device 60, the first vessel end 16 is held in position by a first clamp 72 and the second vessel end 18 is held in position by a second clamp 73. Preferably each of the clamps 72 and 73 are slidably mounted on the bar 66. Herein the clamps 72 and 73 are each in the form of spring biased clamps each having opposed blades 72a and 72b and blade 73a and 73b. The spring biased blades are biased to clamp against opposite sides of the end vessel ends. Thus, the ends of the respective vessels 12 and 14 are juxtapositioned to the anastomosis ring 10 by the coupler device so that the surgeon is then able to quickly grasp the first distal end 16 of the first vessel and pull it through the opening 28 in the anastomosis ring and evert it and impale it on the distal ends of the impaling projections 24. The second vessel may have its distal end 18 then released from the clamp 73 and brought by the surgeon over the now impaled distal end 16 with the intimas at the severed ends of the respective vessels 12 and 14 being in engagement when then are impaled on the protrusions 24 on the exterior surface of the ring thereby holding the lumens of the vessel ends stretched to about the size of bore 28 in the ring to ensure patency.

The clamps may have a portion 80 slidably mounted on the bar 66 with outwardly bent ends 82 which can be flexed toward each other by pinching to open the clamp blades to allow the advancing of the vessel severed ends and releasing. the pinching of the bent ends 82 the clamps will again close. Typically, the blades have a clamping pressure which is quite light, for example, 35 grams or less depending upon the clamp and the vessel, whether it is an artery or a vein as well as to the size of the particular vessel being clamped.

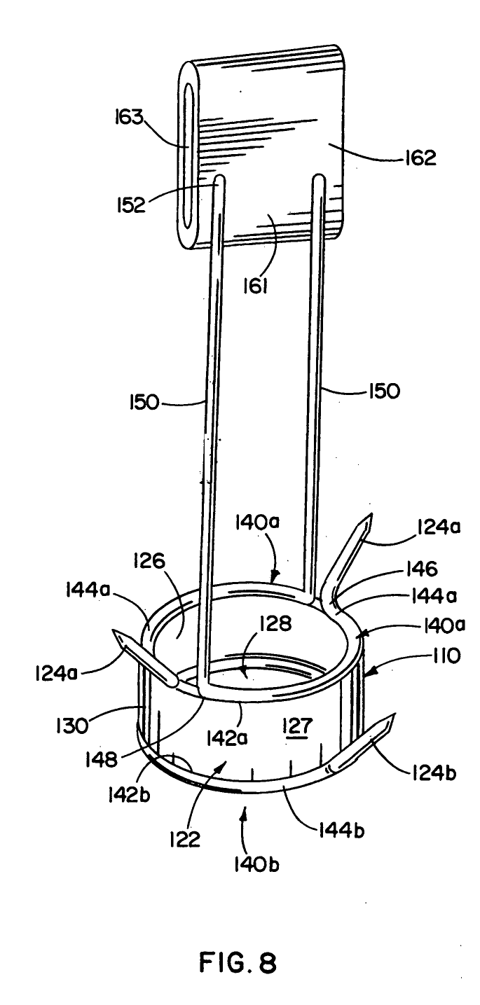

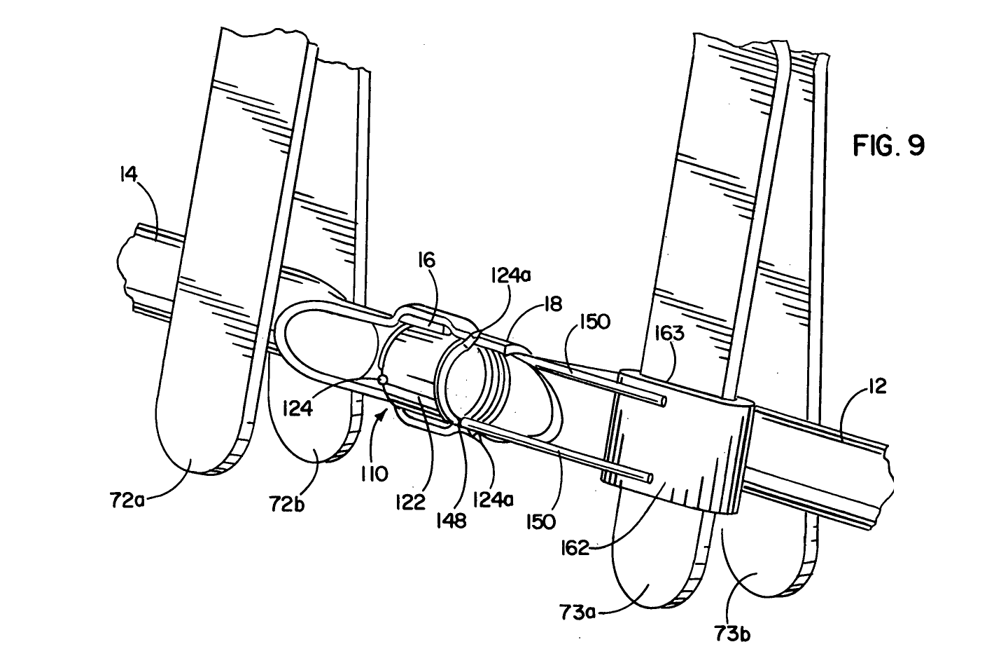

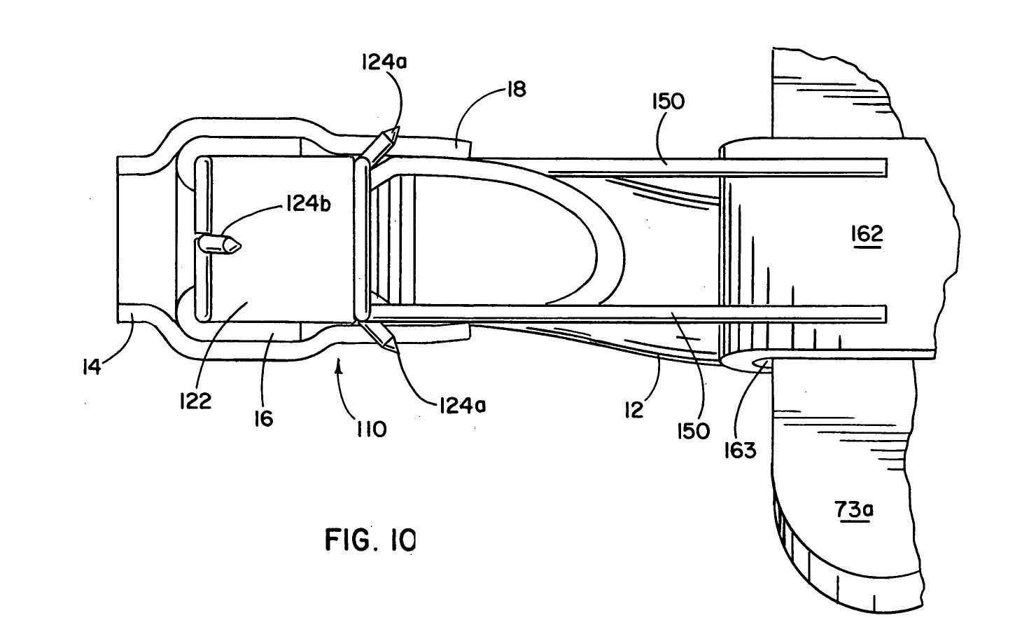

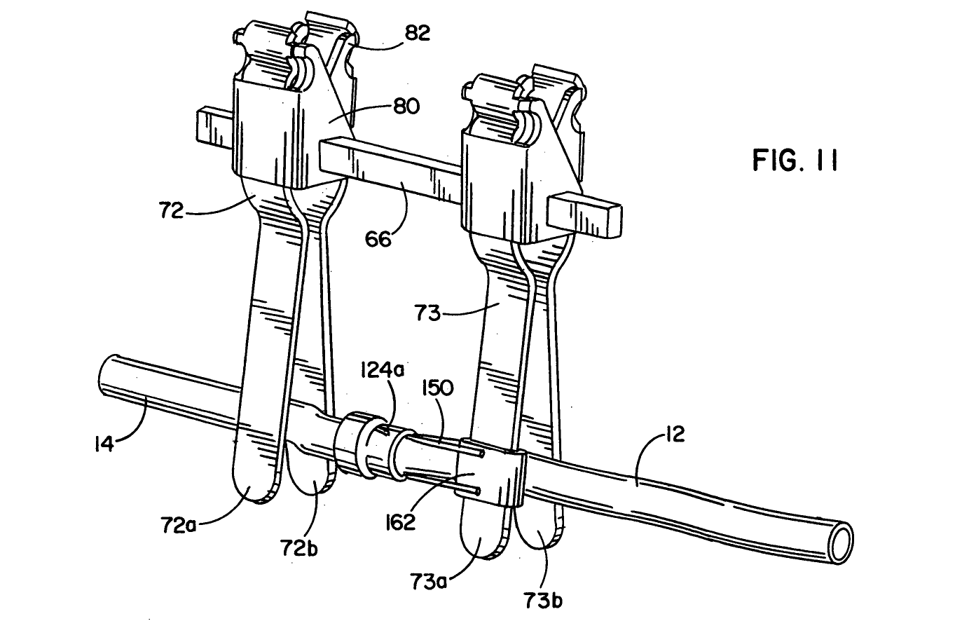

Referring to FIGS. 8-11, an alternative anastomosis device 110 is illustrated. Device 110 is similar to device 10 except that it includes a modified ring holding device 162 to hold the anastomosis ring 110 in position by holding the anastomosis ring 110 to one of the opposed blades or coupling members 72a, 72b, 73a, or 73b of the previously described coupler device 60 rather than the longitudinally extending bar 66.

As shown in FIG. 8, the anastomosis device 110 is also a generally ring-shape and includes a tubular body 122 having a bore or opening 128 therethrough, which the first proximal vessel end 16 of the first vessel 12 may be passed as shown in FIGS. 9 and 11. The tubular body 122 is also made from a biological, compatible material and has an exterior surface 130 and an interior surface 126 that define an annular wall 127 about the central opening 128. As with the previous embodiment, the exterior diameter of the tubular body 122 at the surface 130 is slightly larger than the relaxed exterior diameter of the vessel ends being joined and the interior diameter at the surface 126 is substantially equal to the natural, relaxed exterior diameter of the vessel ends. Preferably, the inside diameter of the device 110 ranges from about 0.85 mm to about 1.2 mm, which allows the device 110 to be used with human finger arteries. Most preferably, the inside diameter is either about 0.85 mm, about 1.0 mm, or about 1.2 mm. Alternatively, the inside diameter may be larger and range from about 2 mm to about 3 mm, which allows the device 110 to be used with the wrist level ulnar or radial arteries.

The device 110 also has a multiple piece construction, where the various components are secured together by a laser weld, for example. For instance, the tubular body 122 includes end walls 142a and 142b to which curved wires 140a and 140b, respectively, are secured. Preferably, the device 110 includes a pair of curved wires 140a secured to the end wall 142a and a pair of curved wires 140b secured to the end wall 142b, which is similar to the previous embodiment.

More specifically, the curved wires 140b are shaped and formed to be an extension of the tubular body 122 and include a curved portion 144b and a protruding projection or portion 124b. The curved wires 140b are similar to the curved wires 40 described in the previous embodiment; accordingly, the discussion on curved wires 40 also applies to curved wires 140b.

The curved wires 140a, on the other hand, are modified to join the anastomosis device 110 to the ring holding device 162. That is, the curved wires 140a have a curved portion 144a and a protruding portion 124a similar to the other embodiments so that wires 140a are also shaped and formed to be an extension of the tubular body 122, but the curved wires 140a also include an extension or connecting member 150 that joins the anastomosis device 110 to the ring holding device 162. The connecting member 150 is readily breakable so that the ring holding device 162 may be detached from the anastomosis device 110.

More specifically, the curved portion 144a includes a distal end 146 from which the projecting portion 124a extends therefrom and a proximal end 148 that is joined to the ring holding device 162 through the extension or support structure 150. Herein, at the proximal end 148, the wire 140a extends outwardly to form the support structure 150. That is, the wire 140a is bent approximately 90° from the curved portion 144a and extends axially outwardly from the tubular body 122 parallel to the central axis extending through the body and is secured to the ring holding device 162 at an end 152 thereof.

To securely hold the anastomosis device 110 to the ring holding device 162, it is preferred that each of the curved wires 140a secured to the end wall 142a include the connecting member 150 so that the ring holding device 162 is secured by a pair of connecting members 150. Each of the connecting members 150 are preferably secured via a laser weld, for example, to an outside surface 161 of the ring holding device 162, but may be secured to the ring holding device 162 in other locations or by other methods.

The ring holding device 162 is spaced from the anastomosis device 110. That is, the support structures 150 space the anastomosis device 110 a predetermined distance from the ring holding device 162 such that when installed on the coupler device 60, the anastomosis device 110 is held in a position between the first and second clamps 72 and 73 to receive the first vessel 12 and second vessel 14. The ring holding device 162 may be a collar, also formed of a biological, compatible material, having a through passage 163 that is generally perpendicular to the bore 128. Preferably, the passage 163 is sized to receive one of the opposed blades 72a, 72b, 73a, or 73b of either the first clamp 72 or the second clamp 73 of the coupling device 60 in a tight, friction fit as shown in FIGS. 9-11.

In use, the anastomosis device 110 only differs from device 10 in how it is detachably secured to the coupler device 60 and how it is detached therefrom. As previously mentioned, the ring holding device 162 holds the anastomosis device 110 in a position so that the vessel ends 16 and 18 may be inserted, everted, and impaled similar to the anastomosis device 10. Accordingly, the previous method of anastomosing the vessels 12 and 14 with device 10 applies to modified device 110

Referring to FIGS. 9-11, to detachably hold the anastomosis device 110 to the coupler device 60, the blade 73a of the clamp 73 is inserted into the through passage 163 of the ring holding device 162. The device 162 is then urged a sufficient distance up the blade 73a so that the anastomosis device 110 is sufficiently secured to the coupling device 60. The vessels 12 and 14 may then be anastomosed as previously described.

To detach the anastomosis device 110 from the coupling device 60 so that the device 110 may remain within the vessels, each of the connecting members 150 are cut using a wire cutting instrument or the like. Preferably, the connecting members 150 are cut near the proximal end 148 of the curved wires 140a. In this manner, the anastomosis device 110 is separated from the connecting members 150, which remain secured to the ring holding device 162, and the device 110 remains to anastomosis the vessels. At the conclusion of the anastomosing procedure, when the vessel ends 16 and 18 are anastomosed, the ring holding device 162 may be removed from the blade 73a and the coupling device 60 may then be reused.

It will be understood that various changes in the details, materials, and arrangements of the parts and components that have been described and illustrated in order to explain the nature of the invention may be made by those skilled in the art within the principle and scope of the invention as expressed in the appended claims.

1. An anastomosis ring for interconnecting ends of living vessels in apposition of the ring, the anastomosis ring comprising:
a tubular member having a hollow bore through which a first end of the vessel may pass;
an exterior surface on the ring to receive an everted first end of the vessel;
a plurality of impaling projections at one end of the tubular member; and
a plurality of impaling projections at the other end of the tubular member.

2. An anastomosis ring in accordance with claim 1 comprising:
end walls on opposite ends of the tubular member; and
a curved member having the impaling projections thereon being secured to at least one end wall of the tubular body.

3. The anastomosis ring of claim 2, further comprising:
an anastomosis ring coupler device having a coupling member; and
a collar spaced from one of the end walls of the tubular member and having a through passage for releasably receiving the coupler device coupling member therein.

4. The anastomosis ring of claim 3, further comprising:
at least one connecting member extending between the one end wall of the tubular member and the collar such that the collar is spaced from the anastomosis ring with the at least one connector configured to be readily breakable for detaching the ring from the collar.

5. The anastomosis ring of claim 4, wherein the curved member comprises first and second curved wires, wherein each of the first and second curved wires comprises:
a curved portion having proximal and distal ends attached to one of the end walls of the tubular member;
an impaling projection extending from the distal end of the curved portion; and
at least one connector member comprises a pair of connector members each extending from the proximal end of the curved portion of one of the wires.

6. An anastomosis ring in accordance with claim 2, wherein the curved member is a curved wire comprising:
a second curved wire having a curved portion secured to the end wall of the tubular member; and
at least one impaling projection at an end of the second curved wire to provide a pair of impaling projections at the end wall.

7. An anastomosis ring in accordance with claim 6, wherein a pair of curved wires are secured to each of the opposite end walls of the tubular member to provide four impaling projections for the anastomosis ring.

8. An anastomosis ring in accordance with claim 7 wherein the impaling projections at the end are positioned to provide impaling projections spaced at 90° from each other in a circumferential direction.

9. An anastomosis ring in accordance with claim 1 wherein the tubular member has an inner diameter from about 0.85 to about 3 mm and about 0.5 to 1.00 mm in length.

10. An anastomosis ring in accordance with claim 6 wherein the wire is about 0.75 mm to 0.125 mm in diameter.

11. An anastomosis ring in accordance with claim 2 comprising:
a laser weld secures the curved member to the tubular member.

12. An anastomosis ring in accordance with claim 1 wherein the impaling projections project forwardly and radially outwardly from the respective end walls of the tubular member.

13. An anastomosis ring in accordance with claim 9 wherein the impaling projections project from the end wall of the tubular member at about a 45° angle.

14. A method of anastomosing first and second vessel ends, the method comprising:
providing an anastomosis ring having a tubular body having at least a impaling projection thereon;
detachably holding the anastomosing ring in a coupler device with the pointed projection pointing toward a first end of the vessel;
entering the first vessel end through a hollow bore in the tubular body and exiting the first vessel end over an exterior surface of the tubular body to evert the first vessel end on the tubular body exterior surface;
impaling the first vessel end on the impaling projections;
drawing the second vessel end over the everted first vessel end and bring their respective intimas into apposition; and
detaching the anastomosis ring from the coupler device to allow the anastomosis ring to remain with a living body without the coupler device.

15. A method in accordance with claim 14 wherein the anastomosis ring is adhered to the coupler and the adherence is broken.

16. A method in accordance with claim 14 wherein the anastomosis ring has a breakable tab secured to the anastomosis device and :the tab is broken off after the first and second vessels are anastomosed.

17. A method in accordance with claim 20, wherein the anastomosis ring includes:
a spaced collar for detachably holding the anastomosis ring to the coupler device;
a connector member projecting outwardly from the tubular body to the collar; and
cutting the connector member after the first and second vessel ends are anastomosed.

18. A method in accordance with claim 17, wherein the connector,: member projects axially outwardly from an end of the attached wire opposite the impaling projection.

19. A method in accordance with claim 14 comprising:
clamping the first vessel end and holding it in position by the coupler device; and
clamping the second vessel end and holding it in position by the coupler device.

20. A method of making an anastomosis ring having impaling projections thereon for anastomosing first and second vessel ends, the method comprising:
providing a tubular body having an annular wall and a pair of opposite end walls;
providing curved members curved to match the curvature of the tubular body;
providing impaling projections on at least one end of the curved members to project outwardly from the tubular body for impaling the vessel ends; and
securing the curved members to the tubular body.

21. A method in accordance with claim 20, wherein at least one of the curved members includes a connector extension projecting outwardly from the tubular body at an end of the curved member opposite the impaling projection.

22. A method in accordance with claim 21, wherein a collar is secured to a distal end of the connector extension and has a passage therethrough; and
fitting a coupler member from an anastomosis ring coupling device through a collar passage for releasably connecting the tubular body to the anastomosis ring coupling device.

23. A method in accordance with claim 22 further comprising securing the coupling member to the collar by a friction fit therebetween.
